(19) United States
(12) Patent Application Publication
(10) Pub. No.: US 2026/0182917 A1
(43) Pub. Date: Jul. 2, 2026

(54) SMART RING

(71) Applicant: Goertek Inc., Weifang (CN)

(72) Inventors: Chengxin ZHANG, Weifang (CN); Junjie Pan, Weifang (CN); Yaocheng Liu, Weifang (CN)

(21) Appl. No.: 19/371,614

(22) Filed: Oct. 28, 2025

Related U.S. Application Data
(63) Continuation of application No. PCT/CN2025/085307, filed on Mar. 27, 2025.

(30) Foreign Application Priority Data
Jun. 27, 2024 (CN) 202410851532.X

(51) Int. Cl. A61B 5/00; A61B 5/024; A61B 5/318; A61B 7/04

(52) U.S. Cl. CPC A61B 5/6826; A61B 5/02427; A61B 5/318; A61B 7/04; A61B 2560/0214; A61B 2560/0462; A61B 2562/0233

(57) ABSTRACT

A smart ring is provided. The smart ring includes a ring body and a detecting assembly, and the ring body provided with a through hole for a finger to pass through. The ring body includes an outer ring and an inner ring, and the outer ring is sleeved on an outside side of the inner ring and encloses with the inner ring to form a mounting space. The mounting space is provided with a circuit board and a battery for powering the circuit board. The detecting assembly includes a VPU acquisition module, a PPG acquisition module, an ECG acquisition module and an optical module mounted on the circuit board and electrically connected to the circuit board.

### CROSS-REFERENCE TO RELATED APPLICATIONS

This is a continuation application of International Application No. PCT/CN2025/085307, filed on Mar. 27, 2025, which claims priority to Chinese Patent Application No. 202410851532.X, filed on Jun. 27, 2024. The disclosures of the above-mentioned applications are incorporated herein by reference in their entireties.

### TECHNICAL FIELD

The present application relates to the technical field of smart wearable devices, and in particular to a smart ring.

### BACKGROUND

Heart sound data can be used to assess health status. Currently, doctors usually collect heart sound data using a stethoscope or an electronic auscultation device. Doctors rely on their experience to assess health status. The accuracy of these assessments depends on the experience of the doctor, and since physical condition of everyone is different, there is no standardized data display. Therefore, the error rate of manual auscultation is high and it is usually impossible to make an accurate judgment.

With the rapid development of technologies such as big data and AI, smart wearable devices can now collect heart sound data, enabling personal health and long-term data collection, thereby collecting sufficient heart sound data for analysis. However, existing smart wearable devices are usually large and uncomfortable to wear for long periods of time, making them inconvenient to wear for long periods of time and therefore unable to continuously collect heart sound data.

### SUMMARY

The main purpose of the present application is to provide a smart ring, aiming solve the problem of existing smart wearable devices, such as large size, poor wearing comfort, and inconvenience in prolonged wear.

In order to achieve the above purpose, the present application provides a smart ring including: a ring body provided with a through hole for a finger to pass through, the ring body includes an outer ring and an inner ring, the outer ring is sleeved on an outside side of the inner ring and encloses with the inner ring to form a mounting space, and the mounting space is provided with a circuit board and a battery for powering the circuit board; and
a detecting assembly, the detecting assembly includes a VPU acquisition module, a PPG acquisition module, an ECG acquisition module and an optical module mounted on the circuit board and electrically connected to the circuit board, the optical module is provided toward the inner ring and is configured to emit light toward the finger passing through the through hole and receive light reflected from the finger to generate an optical signal, the PPG acquisition module is configured to generate a photoplethysmogram based on the optical signal, the ECG acquisition module is configured to acquire an electrical potential signal of the finger and generate an electrocardiogram, and the VPU acquisition module is configured to acquire a heart sound signal.

In an embodiment, an inner wall of the inner ring is provided with elastic bosses at positions corresponds to the VPU acquisition module, the PPG acquisition module, the ECG acquisition module and the optical module, the elastic bosses are protrudingly provided to face inward so that sides of the elastic bosses facing outward form cavities, and the elastic bosses are made of transparent material; and
the VPU acquisition module, the PPG acquisition module, the ECG acquisition module, and the optical module are respectively provided in the cavities of the corresponding elastic bosses, and the elastic bosses are configured to deform outward when squeezed by the finger.

In an embodiment, the VPU acquisition module, the PPG acquisition module, and the ECG acquisition module are distributed at intervals along a circumference direction of the inner ring, and the VPU acquisition module is located between the PPG acquisition module and the ECG acquisition module, when the finger passes through the through hole, the battery and the VPU acquisition module respectively correspond to a finger back and a finger pulp of the finger.

In an embodiment, the optical module includes a photodiode and a light-emitting module, the light-emitting module includes a plurality of light-emitting units, and the plurality of light-emitting units are provided with different spectra and wavelengths; and
the plurality of light-emitting units are provided at the circuit board at intervals along the circumference direction of the inner ring and are configured to emit light toward the finger, and the photodiode is configured to receive light reflected from the finger to generate the optical signal.

In an embodiment, an outer periphery of each photodiode is provided with a retaining wall, and an opening for light to pass through is formed at a side of the retaining wall facing the through hole.

In an embodiment, the outer ring, the inner ring, and the circuit board are concentrically provided circular rings, the light-emitting units include a green light-emitting diode (LED) and a red LED, a central angle formed between the green LED and the photodiode is 25° to 45°, and a central angle formed between the red LED and the photodiode is 35° to 65°.

In an embodiment, two photodiodes are provided, the two photodiodes are provided at intervals along the circumference direction of the inner ring, and the light-emitting module is located between the two photodiodes.

In an embodiment, a photodiode is provided, the light-emitting module is located on a side of the circuit board, when the finger passes through the through hole, a line between the finger pulp and the finger back of the finger is a symmetry axis, and the photodiode and the light-emitting module are symmetrically provided about the symmetry axis.

In an embodiment, a photodiode is provided, and the photodiode is located between any two adjacent light-emitting units in the light-emitting module.

In an embodiment, a cross-sectional dimension of the boss is decreased gradually from an outside to an inside, and a protrusion height of the boss is greater than or equal to 1 mm.

In an embodiment, the outer ring protrudes outward at a position corresponding to the VPU acquisition module to form a protrusion, so that a side of the protrusion facing inward forms an accommodating cavity, and the VPU acquisition module is encapsulated in the accommodating cavity and abuts against the outer ring.

In an embodiment, an outer side of the outer ring is provided with a hard contact point protruding outward from the outer ring, so that a side of the hard contact facing inward forms a mounting cavity for accommodating the VPU acquisition module, a rigid frame is provided in the outer ring, an elastic member is mounted on the rigid frame, and the contact shell is configured to push the VPU acquisition module and drive the elastic member to compress when subjected to pressure

In technical solution of the present application, by conducting the VPU acquisition module, the PPG acquisition module, the ECG acquisition module, and the optical module with the circuit board, and then covering and shielding the above modules with the inner ring, the user will not feel discomfort when wearing the smart ring, thereby improving wearing comfort. The PPG acquisition module and the optical module work together to detect and generate a photoplethysmogram, and the ECG acquisition module generates an electrocardiogram, thereby providing real-time monitoring of the user. When heart sound signals are required, the ring is attached to the chest of the user, allowing the VPU acquisition module to collect heart sound signals. The present application integrates multiple heart sound detecting modules into a smart ring. The smart ring is small in size, comfortable to wear, and easy to carry for a long time. Therefore, it is convenient to collect and analyze the heart sound data of the user at various times and in various states over a long period of time, thereby improving the accuracy of the analysis of the heart sound data of the user.

### BRIEF DESCRIPTION OF THE DRAWINGS

In order to illustrate the technical solutions in the embodiments of the present application or in the related art more clearly, the following briefly introduces the accompanying drawings required for the description of the embodiments or the related art. Obviously, the drawings in the following description are only part of embodiments of the present application. For those skilled in the art, other drawings can also be obtained according to the structures shown in these drawings without any creative effort.

FIG. 1 is a schematic structural diagram of a smart ring according to an embodiment of the present application.

FIG. 2 is a schematic cross-sectional structural diagram of the smart ring according to an embodiment of the present application.

FIG. 3 is a schematic structural diagram of the smart ring according to another embodiment of the present application.

FIG. 4 is a schematic structural diagram of a circuit board of the smart ring according to another embodiment of the present application.

FIG. 5 is a schematic structural diagram of an inner ring of the smart ring according to another embodiment of the present application.

FIG. 6 is a schematic side cross-sectional structural diagram of the smart ring according to an embodiment of the present application.

FIG. 7 is a schematic side cross-sectional structural diagram of the smart ring according to another embodiment of the present application.

FIG. 8 is a schematic diagram of a partial structure of the smart ring according to an embodiment of the present application.

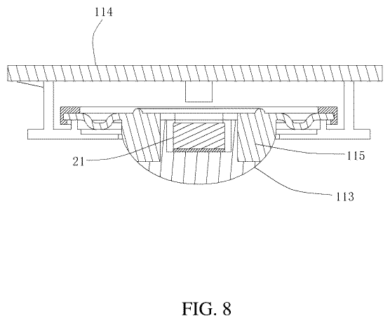

FIG. 9 is a schematic diagram of a positional relationship of optical modules of the smart ring according to an embodiment of the present application.

The realization of the objective, functional characteristics, and advantages of the present application are further described with reference to the accompanying drawings.

### DETAILED DESCRIPTION OF THE EMBODIMENTS

The technical solutions of the embodiments of the present application will be described in more detail below with reference to the accompanying drawings. It is obvious that the embodiments to be described are only some rather than all of the embodiments of the present application. All other embodiments obtained by persons skilled in the art based on the embodiments of the present application without creative efforts shall fall within the scope of the present application.

It should be noted that if there are directional indications, such as up, down, left, right, front, back, etc., involved in the embodiments of the present application, the directional indications are only used to explain a certain posture as shown in the accompanying drawings. If the specific posture changes, the directional indication also changes accordingly.

In addition, if there are descriptions related to “first”, “second”, etc. in the embodiments of the present application, the descriptions of “first”, “second”, etc. are only for the purpose of description, and should not be construed as indicating or implying relative importance or implicitly indicating the number of technical features indicated. Thus, a feature delimited with “first”, “second” may expressly or implicitly include at least one of these features. Besides, the meaning of “and/or” appearing in the application includes three parallel scenarios. For example, “A and/or B” includes only A, or only B, or both A and B. In addition, the technical solutions between the various embodiments can be combined with each other, but must be based on the realization by those skilled in the art. When the combination of technical solutions is contradictory or cannot be realized, it should be considered that the combination of such technical solutions does not exist or fall within the scope of the present application.

With the rapid development of technologies such as big data and AI, smart wearable devices can now collect heart sound data, enabling personal health and long-term data collection, thereby collecting sufficient heart sound data for analysis. However, existing smart wearable devices are usually large and uncomfortable to wear for long periods of time, making them inconvenient to wear for long periods of time and therefore unable to continuously collect heart sound data.

In order to solve the above problems, the present application provided a smart ring 100.

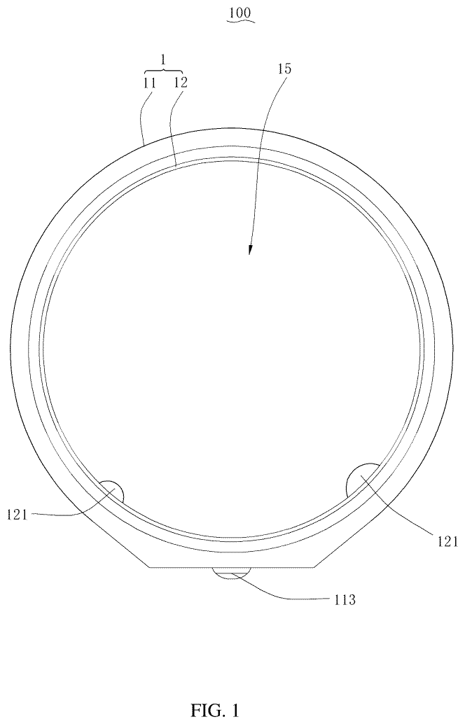

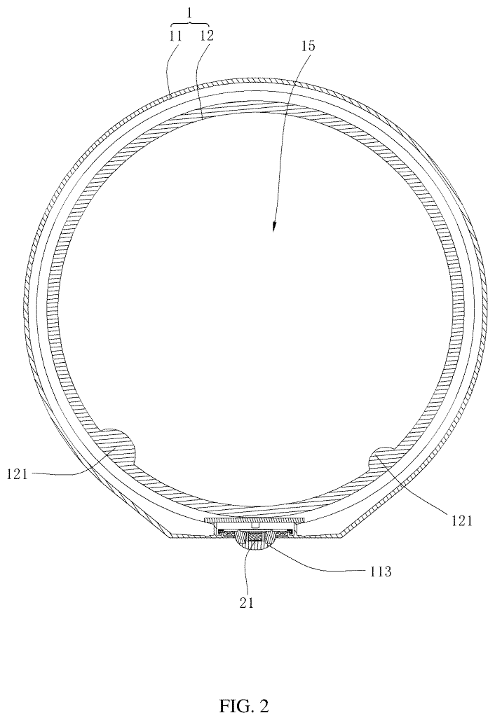

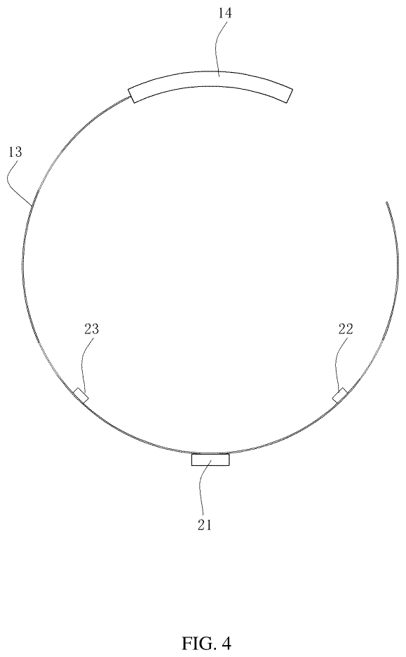

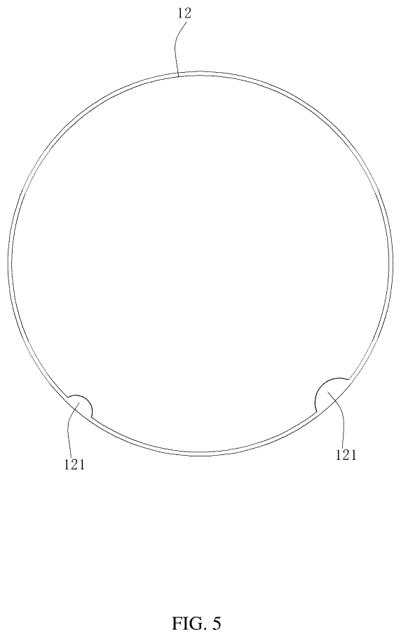

Referring to FIG. 1, FIG. 2, FIG. 4, and FIG. 5, in an embodiment of the present application, the smart ring 100 includes a ring body 1 and a detecting assembly 2, and the ring body 1 is provided with a through hole 15 for a finger to pass through. The ring body 1 includes an outer ring 11 and an inner ring 12, the outer ring 11 is sleeved on an outside side of the inner ring 12 and encloses with the inner ring 12 to form a mounting space, and the mounting space is provided with a circuit board 13 and a battery 14 for powering the circuit board 13. The detecting assembly 2 includes a VPU acquisition module 21, a PPG acquisition module 22, an ECG acquisition module 23 and an optical module 24 mounted on the circuit board 13 and electrically connected to the circuit board 13. The optical module 24 is provided toward the inner ring 12 and is configured to emit light toward a finger passing through the through hole 15 and to receive light reflected from the finger to generate an optical signal. The PPG acquisition module 22 is configured to generate a photoplethysmogram based on the optical signal. The ECG acquisition module 23 is configured to acquire the potential signal of the finger and generate an electrocardiogram. The VPU acquisition module 21 is configured to acquire heart sound signals.

It should be noted that the VPU (heart sound signal acquisition) module, PPG (photoplethysmography) acquisition module and ECG (electrocardiogram) acquisition module are all commonly used modules for heart sound data acquisition in the related art, and their working principles can be found in the related art. At the same time, the outer ring 11 is usually made of rigid material to provide support for the overall shape of the ring, and the inner ring 12 is usually made of flexible material so that the inner ring 12 can be deformed and close to the finger of the user when the user wears the ring, thereby improving the experience and comfort of the user. In an embodiment, the flexible material can be soft rubber materials such as silicone, TPU, TPE, or soft glue materials.

In technical solution of the present application, by conducting the VPU acquisition module 21, the PPG acquisition module 22, the ECG acquisition module 23, and the optical module 24 with the circuit board 13, and then covering and shielding the above modules with the inner ring 12, the user will not feel discomfort when wearing the smart ring 100, thereby improving wearing comfort. The PPG acquisition module 22 and the optical module 24 work together to detect and generate a photoplethysmogram while the ECG acquisition module 23 generates an electrocardiogram, thereby providing real-time monitoring of the user. When heart sound signals are needed, the ring is attached to the chest of the user, allowing the VPU acquisition module 21 to collect heart sound signals. The present application integrates multiple heart sound detecting modules in the smart ring 100. The smart ring 100 is small in size and highly comfortable to wear, making it easy to carry for long periods of time. This facilitates the long-term collection and analysis of the heart sound data of the user at various times and in various states, thereby improving the accuracy of the analysis of the heart sound data of the user.

In an embodiment, after the circuit board 13 is mounted on the outer ring 11, excess cavity area between the outer ring 11 and the circuit board 13 is filled by a glue filling process to fix the circuit board 13 and the outer ring 11. At the same time, after the glue solidifies, it can also protect the circuit board 13 and the multiple modules provided at the circuit board 13, thereby improving safety and stability.

Please refer to FIG. 4 and FIG. 5, in an embodiment, the inner wall of the inner ring 12 is provided with elastic bosses 121 at the positions corresponds to the VPU acquisition module 21, the PPG acquisition module 22, the ECG acquisition module 23 and the optical module 24. The elastic boss 121 is protrudingly provided to face inward so that sides of the elastic bosses 121 facing outward form cavities, and the quality of elastic bosses 121 are made of transparent material. The VPU acquisition module 21, the PPG acquisition module 22, the ECG acquisition module 23 and the optical module 24 are respectively provided in the cavities of the corresponding elastic bosses 121. The elastic boss 121 is configured to deform outward when squeezed by the finger. By setting the cavity of the elastic boss 121, the VPU acquisition module 21, the PPG acquisition module 22, the ECG acquisition module 23 and the optical module 24 are brought into closer contact with the finger of the user inserted into the through hole 15, thereby improving the accuracy of data detecting. At the same time, since the elastic boss 121 is made of elastic material, when the finger of the user is inserted into the through hole 15, the elastic boss 121 will be squeezed and deformed, which will not affect the finger of the user, thereby improving the wearing comfort of the user.

In an embodiment, the material hardness of the inner ring 12 is 30-70 (Shore hardness), so that the inner ring 12 can produce appropriate deformation to match the finger contours of different users and improve wearing comfort. The material of the elastic boss 121 is TPU, TPE, silicone, rubber and fluororubber, etc., with a hardness of 30-45 (Shore hardness). The hardness of the material of the elastic boss 121 is less than that of other areas of the inner ring 12 to reduce wearing discomfort.

In an embodiment, the VPU acquisition module 21, the PPG acquisition module 22, and the ECG acquisition module 23 are distributed at intervals along the circumference direction of the inner ring 12, and the VPU acquisition module 21 is located between the PPG acquisition module 22 and the ECG acquisition module 23. When the finger passes through the through hole, the battery 14 and the VPU acquisition module 21 respectively correspond to the finger back and the finger pulp of the finger. The battery 14 is provided near the center of the finger back of the finger, and the VPU acquisition module 21 is provided corresponding to the finger pulp of the finger. At the same time, the PPG acquisition module 22 and the ECG acquisition module 23 are respectively located at both end of the VPU acquisition module 21. This ensures that the overall weight of the ring is evenly distributed, and the center of gravity remains at the center of the smart ring 100, improving the wearing comfort of the user.

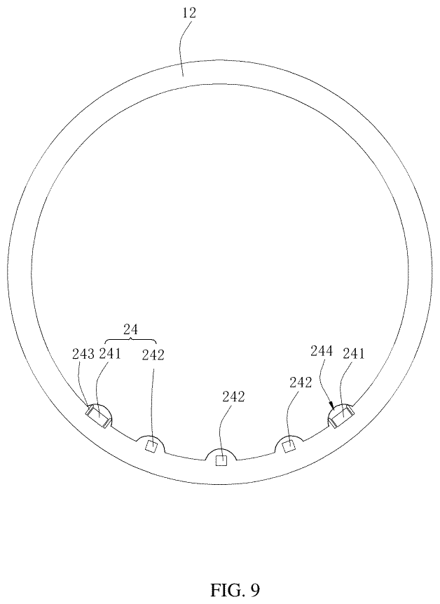

Referring to FIG. 9, in an embodiment, the optical module 24 includes a photodiode 241 and a light-emitting module. The light-emitting module includes a plurality of light-emitting units 242, and the plurality of light-emitting units are provided with different spectra and wavelengths. The plurality of light-emitting units 242 provided at the circuit board 13 at intervals along the circumference direction of the inner ring 12 and are configured to emit light toward the finger. The photodiode 241 receives the light reflected from the finger to generate an optical signal. By providing light-emitting units 242 with different spectra and wavelengths, different heart sound data can be detected. In an embodiment, when performing PPG remote measurement, heart rate is typically measured using a green light-emitting diode (LED) (wavelength approximately 520 nm-540 nm), while blood oxygen saturation is typically measured using a combination of a red LED (typical wavelength 660 nm) and an infrared LED (typical wavelength 940 nm) to achieve simultaneous detection of multiple data. The light reflected from the blood vessels of the finger is then received by the photodiode 241, and an optical signal is generated, which facilitates subsequent analysis of specific heart sound data based on the optical signal.

In an embodiment, the outer periphery of each photodiode 241 is provided with a retaining wall 243, and an opening 244 for light to pass through is formed at the side of the retaining wall 243 facing the through hole 15. The photodiode 241 is circumferentially blocked by the retaining wall 243 to prevent the light from the light-emitting unit 242 or the ambient light from affecting the accuracy of the optical signal, leaving only an opening 244 toward the center of the through hole 15, so that only the light reflected back from the blood vessel can be collected by the photodiode 241, thereby maximizing the detection accuracy.

In an embodiment, the outer ring 11, the inner ring 12 and the circuit board 13 are concentrically provided circular rings, and the light-emitting unit 242 includes a green LED and a red LED. The central angle formed by the green LED and the photodiode 241 is 25° to 45°, and the central angle formed by the red LED and the photodiode 241 is 35° to 65°. The central angle formed between the green LED and the photodiode 241 is small because green light is mainly configured to detect blood flow changes in shallow tissues to achieve heart rate monitoring, such as the skin and microvessels under the epidermis. The smaller angle can ensure that the detected reflected light mainly comes from these shallow tissues. The red light and the infrared light have high tissue penetrability and can pass through the skin and deeper tissues, such as the muscle layer, to detect blood flow changes in deeper blood vessels. The larger angle helps to capture light reflected or transmitted back from these deep tissues, thereby obtaining blood oxygen saturation through changes in blood flow in blood vessels in deep tissues.

In an embodiment, two photodiodes 241 are provided, and the two photodiodes 241 are provided at intervals along the circumference direction of the inner ring 12, and the light emitting module is located between the two photodiodes 241.

In another embodiment, a photodiode 241 is provided, and the light-emitting module is located on a side of the circuit board 13. When a finger passes through the through hole, the line between the finger pulp and finger back of the finger is a symmetry axis, and the photodiode 241 and the light-emitting module are symmetrically provided about the symmetry axis.

In another embodiment, a photodiode 241 is provided, and the photodiode 241 is located between any two adjacent light-emitting units 242 in the light-emitting module.

The number and position relationship between the photodiodes 241 and the light emitting module can be adjusted according to actual needs, but it is necessary to ensure that the overall arrangement is symmetrical with the line between the finger pulp and the finger back of the finger as the axis of symmetry to ensure the position of the center of gravity.

In an embodiment, the cross-sectional dimension of the boss is decreased gradually from an outside to an inside, and the height of the boss is greater than or equal to 1 mm. The cross-sectional dimension of the cavity is gradually decreased toward the finger of the user, reducing the contact area between the boss and the finger of the user, thereby minimizing the foreign body sensation felt by the finger of the user and improving wearing comfort. In an embodiment, the cross-sectional dimension of the boss can be arc-shaped or trapezoidal, with the cross-sectional dimension tapering toward the finger of the user while leaving ample space for accommodating the light-emitting unit. The raised design facilitates deformation towards the side away from the finger after being pressed, reducing the foreign body sensation.

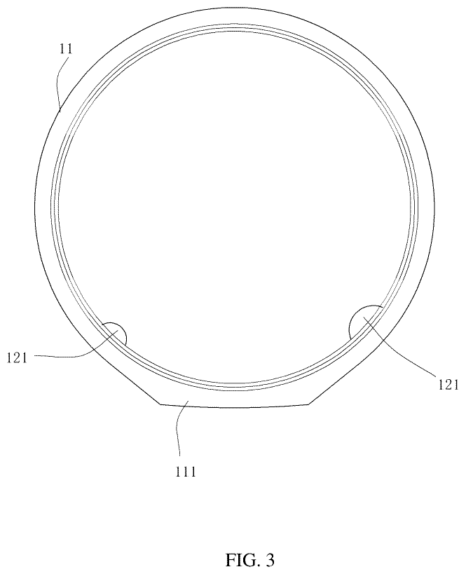

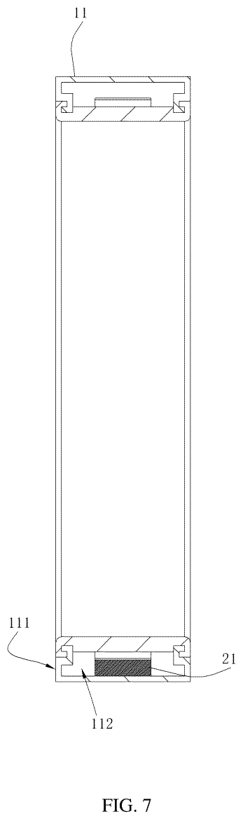

Referring to FIG. 3 and FIG. 7, in an embodiment, the outer ring 11 protrudes outward at a position corresponding to the VPU acquisition module 21 to form a protrusion 111, so that the side of the protrusion 111 facing inward forms an accommodating cavity 112, and the VPU acquisition module 21 is encapsulated within the accommodating cavity 112 and abuts against the outer ring 11. By setting the protrusion 111, the distance between the VPU acquisition module 21 and the chest is closer when the VPU acquisition module 21 is close to the chest of the user, with only one layer of shell separating them, thereby improving the detection accuracy of the VPU acquisition module 21.

In an embodiment, the width of the protrusion along the circumference direction of the ring is 10 mm to 20 mm, which is smaller than the width of the finger of the user, so as to improve wearing comfort.

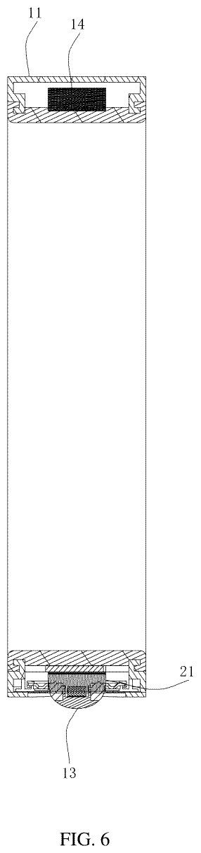

Please refer to FIG. 1, FIG. 2 and FIG. 6, in another embodiment, an outer side of the outer ring 11 is provided with a hard contact point 113 protruding outward from the outer ring 11, so that the side of the hard contact point 113 facing inward forms a mounting cavity for accommodating the VPU acquisition module 21. A rigid frame 114 is provided in the outer ring 11, and an elastic member 115 is mounted on the rigid frame 114. The contact shell is configured to push the VPU acquisition module 21 and drive the elastic member 115 to compress when subjected to pressure. The mounting cavity formed by the hard contact point 113 allows the VPU acquisition module 21 to be exposed on the surface of the outer ring 11, so that the distance between the VPU acquisition module 21 and the chest is closer when it is close to the chest of the user, with only one layer of shell separating them, thereby improving the detecting accuracy of the VPU acquisition module 21. At the same time, the setting of the elastic member 115 makes it possible for the elastic member 115 to compress after the hard contact point 113 contacts the chest of the user, driving the hard contact point 113 to move inward, leaving a movable margin to avoid damage due to pressure. Or in daily life, when the hand of the user is placed on the table, the hard contact point 113 moves inward to reduce the foreign body sensation of the user.

The rigid frame 114 is made of stainless steel, aluminum alloy, magnesium alloy, titanium alloy, etc., and configured to support and fix the elastic member 115. The elastic member 115 is made of TPU, TPE, silicone, rubber and fluororubber, etc., with a hardness of 30 to 65 (Shore hardness), so that the elastic member 115 is compressed inside after being compressed to provide a movement space for the hard contact point 113. The material of the hard contact point 113 is a rigid material such as titanium alloy, magnesium alloy, aluminum alloy or a silicone material with a hardness of 50 to 70 (Shore hardness). After the VPU acquisition module 21 is mounted in the mounting cavity of the hard contact point 113, glue is filled in through a glue-filling process so that the glue can fix and protect the VPU acquisition module 21 after solidification.

The above descriptions are only embodiments of the present application, and are not intended to limit the scope of the present application. Under the inventive concept of the present application, any equivalent structural transformations made by using the contents of the description and drawings of the present application, or direct/indirect applications in other related technical fields are included in the scope of the present application.

What is claimed is:

1. A smart ring, comprising:
a ring body provided with a through hole for a finger to pass through, wherein the ring body comprises an outer ring and an inner ring, the outer ring is sleeved on an outside side of the inner ring and encloses with the inner ring to form a mounting space, and the mounting space is provided with a circuit board and a battery for powering the circuit board; and
a detecting assembly, wherein the detecting assembly comprises a VPU acquisition module, a PPG acquisition module, an ECG acquisition module and an optical module mounted on the circuit board and electrically connected to the circuit board, the optical module is provided toward the inner ring and is configured to emit light toward the finger passing through the through hole and receive light reflected from the finger to generate an optical signal, the PPG acquisition module is configured to generate a photoplethysmogram based on the optical signal, the ECG acquisition module is configured to acquire an electrical potential signal of the finger and generate an electrocardiogram, and the VPU acquisition module is configured to acquire a heart sound signal.

2. The smart ring according to claim 1, wherein:
an inner wall of the inner ring is provided with elastic bosses at positions corresponds to the VPU acquisition module, the PPG acquisition module, the ECG acquisition module and the optical module, the elastic bosses are protrudingly provided to face inward so that sides of the elastic bosses facing outward form cavities, and the elastic bosses are made of transparent material; and
the VPU acquisition module, the PPG acquisition module, the ECG acquisition module, and the optical module are respectively provided in the cavities of the corresponding elastic bosses, and the elastic bosses are configured to deform outward when squeezed by the finger.

3. The smart ring according to claim 2, wherein the VPU acquisition module, the PPG acquisition module, and the ECG acquisition module are distributed at intervals along a circumference direction of the inner ring, and the VPU acquisition module is located between the PPG acquisition module and the ECG acquisition module, when the finger passes through the through hole, the battery and the VPU acquisition module respectively correspond to a finger back and a finger pulp of the finger.

4. The smart ring according to claim 3, wherein:
the optical module comprises a photodiode and a light-emitting module, the light-emitting module comprises a plurality of light-emitting units, and the plurality of light-emitting units are provided with different spectra and wavelengths; and
the plurality of light-emitting units are provided at the circuit board at intervals along the circumference direction of the inner ring and are configured to emit light toward the finger, and the photodiode is configured to receive light reflected from the finger to generate the optical signal.

5. The smart ring according to claim 4, wherein an outer periphery of each photodiode is provided with a retaining wall, and an opening for light to pass through is formed at a side of the retaining wall facing the through hole.

6. The smart ring according to claim 4, wherein the outer ring, the inner ring, and the circuit board are concentrically provided circular rings, the light-emitting units comprise a green light-emitting diode (LED) and a red LED, a central angle formed between the green LED and the photodiode is 25° to 45°, and a central angle formed between the red LED and the photodiode is 35° to 65°.

7. The smart ring according to claim 4, wherein two photodiodes are provided, the two photodiodes are provided at intervals along the circumference direction of the inner ring, and the light-emitting module is located between the two photodiodes.

8. The smart ring according to claim 4, wherein a photodiode is provided, the light-emitting module is located on a side of the circuit board, when the finger passes through the through hole, a line between the finger pulp and the finger back of the finger is a symmetry axis, and the photodiode and the light-emitting module are symmetrically provided about the symmetry axis.

9. The smart ring according to claim 4, wherein a photodiode is provided, and the photodiode is located between any two adjacent light-emitting units in the light-emitting module.

10. The smart ring according to claim 2, wherein a cross-sectional dimension of the boss is decreased gradually from an outside to an inside, and a protrusion height of the boss is greater than or equal to 1 mm.

11. The smart ring according to claim 1, wherein the outer ring protrudes outward at a position corresponding to the VPU acquisition module to form a protrusion, so that a side of the protrusion facing inward forms an accommodating cavity, and the VPU acquisition module is encapsulated in the accommodating cavity and abuts against the outer ring.

12. The smart ring according to claim 1, wherein an outer side of the outer ring is provided with a hard contact point protruding outward from the outer ring, so that a side of the hard contact facing inward forms a mounting cavity for accommodating the VPU acquisition module, a rigid frame is provided in the outer ring, an elastic member is mounted on the rigid frame, and the contact shell is configured to push the VPU acquisition module and drive the elastic member to compress when subjected to pressure.
